(19) United States
(12) Patent Application Publication
(10) Pub. No.: US 2010/0114325 A1
(43) Pub. Date: May 6, 2010

(54) Prophylactic Pancreatic Stent

(75) Inventors: Xiujiang Yang, Shanghai (CN); Wenfeng Lu, Irvine, CA (US)

Correspondence Address: BRINKS HOFER GILSON & LIONE/CHICAGO/COOK, PO BOX 10395, CHICAGO, IL, 60610, US

(73) Assignee: Wilson-Cook Medical Inc., Winston-Salem, NC (US)

(21) Appl. No.: 12/582,036

(22) Filed: Oct. 20, 2009

Related U.S. Application Data
(60) Provisional application No. 61/107,502, filed on Oct. 22, 2008.

(51) Int. Cl. A61F 2/04

(52) U.S. Cl. 623/23.7

(57) ABSTRACT

A stent and a method for implanting a stent for prophylactically protecting a duct are provided. The stent includes a non-expandable, generally tubular body having a proximal portion and a distal portion, a lumen extending through at least a portion of the body, a distal opening in the distal portion in fluid communication with the lumen and a proximal opening in the proximal portion in fluid communication with the lumen. The stent further includes a cap portion operably connected to the proximal portion of the body, the cap portion is movable between a non-expanded configuration and an expanded configuration. The non-expanded configuration has a reduced diameter to facilitate advancement into the patient and the expanded configuration has an expanded diameter configured to protect an opening of the internal bodily duct so as to prevent an unintentional entry of an elongate medical device through the opening and into the passageway of the internal bodily duct.

### RELATED APPLICATIONS

This application claims the benefit of U.S. Provisional Application No. 61/107,502, filed Oct. 22, 2008, which is incorporated herein in its entirety.

### TECHNICAL FIELD

This invention generally relates to stents that are implantable in a vessel or duct within the body of a patient, and in particular to stents that may be used to protect the vessel or duct during a medical procedure.

### BACKGROUND

Minimally invasive medical procedures are performed in various vessels and ducts in the body. In some procedures, access to a vessel or duct may be needed to place prosthetic devices, to obtain a biopsy sample or to otherwise remove tissue material or other substances.

Minimally invasive medical procedures are frequently performed in the biliary system. Entry to the biliary system is through the duodenum into Ampulla of Vater leading to the branch for the pancreatic duct or the common bile duct (CBD) and the gall bladder. In most procedures, entry to the CBD is achieved by cannulation using a sphincterotome catheter or an ERCP catheter to gain entry to the bile duct. Typically, a wireguide is preloaded within a lumen of the catheter. Once successful cannulation is performed, the physician may advance the wireguide further into the CBD. The sphincterotome or ERCP catheter may be withdrawn and another device may be advanced over the wireguide into the CBD, such as a biliary stent, a stone extraction balloon or basket, etc. Advancing a catheter/wireguide for entry into the bile duct can be very difficult due to the anatomy of the papilla, the CBD and the pancreatic duct. Often, a physician may accidentally advance the catheter/wireguide into the pancreatic duct. For an inexperienced endoscopist, multiple entries into the pancreatic duct may occur. Single or repeated misplacement of the catheter/wireguide into the pancreatic duct may cause edema or worse trauma that may hinder pancreatic fluid drainage. Injury to the pancreatic duct can lead to post procedural pancreatitis, a very painful and potentially fatal illness.

There is a need for a method and a stent that can prophylactically reduce or prevent acute pancreatitis when minimally invasive medical procedures are preformed in the biliary system. In addition, the prophylactic stent may be helpful for inexperienced endoscopists and to reduce the time and complexity of procedures within the biliary system. The stent may be atraumatically placed within a pancreatic duct and remain in place without causing aggravation to the ductal tissue and prevent accidental entry into the pancreatic duct when the bile duct is the intended target. The stent may be temporarily placed and can be removed with little damage or additional irritation to the duct.

### SUMMARY OF THE INVENTION

Accordingly, it is an object of the present invention to provide a method and a stent having features that resolve or improve on one or more of the above-described drawbacks.

The foregoing object is obtained in one aspect of the present invention by providing a stent for prophylactically protecting a duct are provided. The stent includes a non-expandable, generally tubular body having a proximal portion and a distal portion, a lumen extending through at least a portion of the body, a distal opening in the distal portion in fluid communication with the lumen and a proximal opening in the proximal portion in fluid communication with the lumen. The stent further includes a cap portion operably connected to the proximal portion of the body, the cap portion is movable between a non-expanded configuration and an expanded configuration. The non-expanded configuration has a reduced diameter to facilitate advancement into the patient and the expanded configuration has an expanded diameter configured to protect an opening of the internal bodily duct so as to prevent an unintentional entry of an elongate medical device through the opening and into the passageway of the internal bodily duct.

In another aspect of the present invention, a method for implanting a prophylactic stent in a duct is provided. The method includes entering a branched ductal system with a wireguide, advancing the wireguide into a first duct of the branched ductal system and advancing a stent over the wireguide. The stent includes a non-expandable, generally tubular body having a proximal portion and a distal portion, a lumen extending through at least a portion of the body, a distal opening in the distal portion in fluid communication with the lumen and a proximal opening in the proximal portion in fluid communication with the lumen. The stent further includes a cap portion operably connected to the proximal portion of the body, the cap portion is movable between a non-expanded configuration and an expanded configuration. The method further includes implanting at least a portion of the distal portion of the stent in the first duct so that the cap portion is in the expanded configuration and protecting an opening in the first duct to inhibit the wireguide from reentering the first duct.

### BRIEF DESCRIPTION OF THE DRAWINGS

FIG. 1A is a perspective view of a stent according to the present invention;

FIG. 1B is a perspective view of an alternate embodiment of a stent according to the present invention;

FIG. 2 is an alternative perspective view of the stent shown in FIG. 1A;

FIG. 3 is a side view of a stent according to an embodiment of the present invention having a plurality of openings in the body;

FIG. 4 is a side view of a stent according to an embodiment of the present invention having a plurality of openings around the cap;

FIG. 5A is a top perspective view of the cap and body of the stent according to the present invention showing a plurality of openings;

FIG. 5B is a side view of the cap and the body of the stent according to an alternative embodiment of the present invention showing a plurality of openings;

FIG. 6A is a side sectional view of an introducer with the stent of FIG. 1A therein for delivery;

FIG. 6B is a side sectional view of the introducer shown in FIG. 6A with the stent released from the introducer and the cap expanded;

FIG. 7A is a side sectional view of the introducer with the stent of FIG. 1B therein for delivery;

FIG. 7B is a side sectional view of the introducer shown in FIG. 7A with the stent released from the introducer and the cap expanded and the proximal portion in the angled configuration;

FIG. 8 is a diagrammatic view of a wireguide advanced through the pancreatic duct;

FIG. 9 is a diagrammatic view of the placement of the prophylactic stent within the pancreatic duct;

FIG. 10 is a diagrammatic view of the stent within the pancreatic duct with the cap protecting the entry to the pancreatic duct; and

FIG. 11 is a diagrammatic view of the wireguide entering the biliary duct.

### DETAILED DESCRIPTION OF THE EMBODIMENTS

The invention is described with reference to the drawings in which like elements are referred to by like numerals. The relationship and functioning of the various elements of this invention are better understood by the following detailed description. However, the embodiments of this invention are not limited to the embodiments illustrated in the drawings. It should be understood that the drawings are not to scale, and in certain instances details have been omitted which are not necessary for an understanding of the present invention, such as conventional fabrication and assembly.

As used in the specification, the terms proximal and distal should be understood as being in the terms of a physician delivering the stent to a patient. Hence the term “distal” means the portion of the stent that is farthest from the physician and the term “proximal” means the portion of the stent that is nearest to the physician.

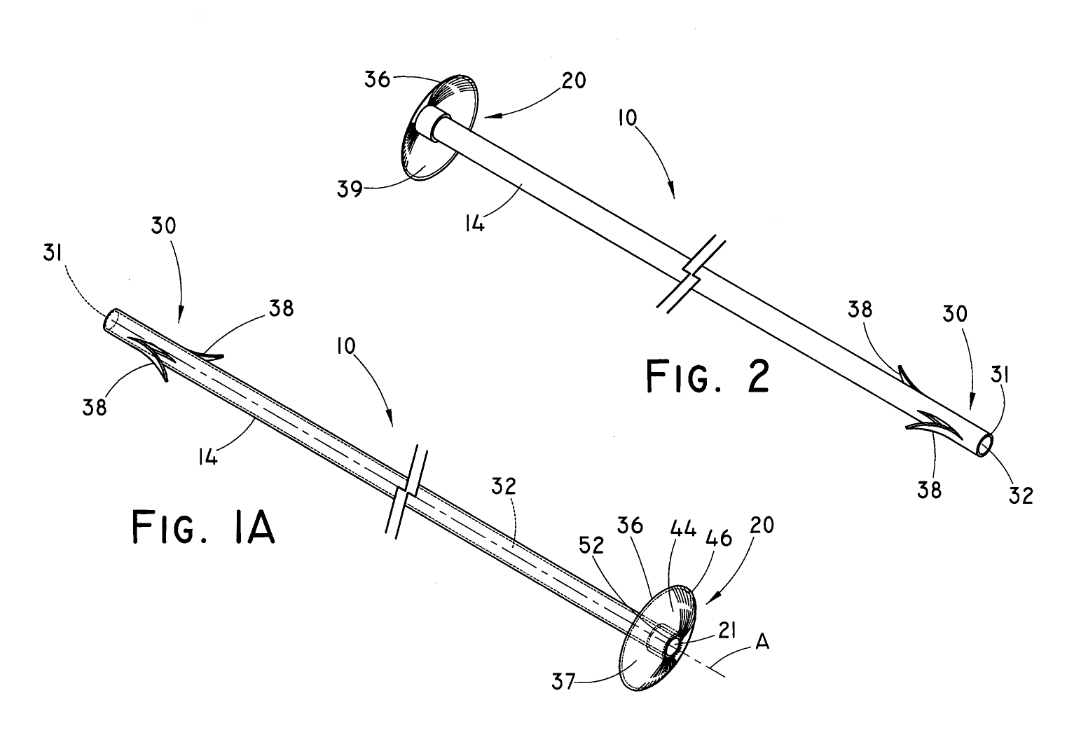

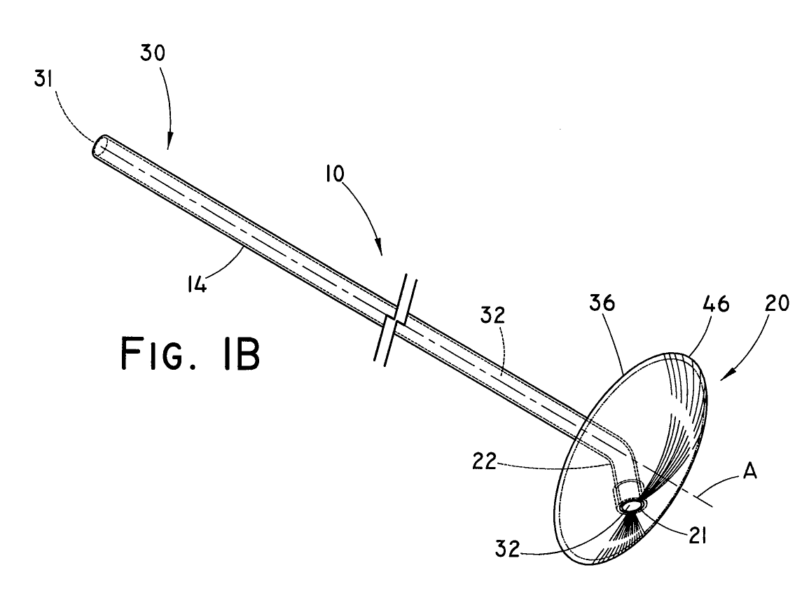

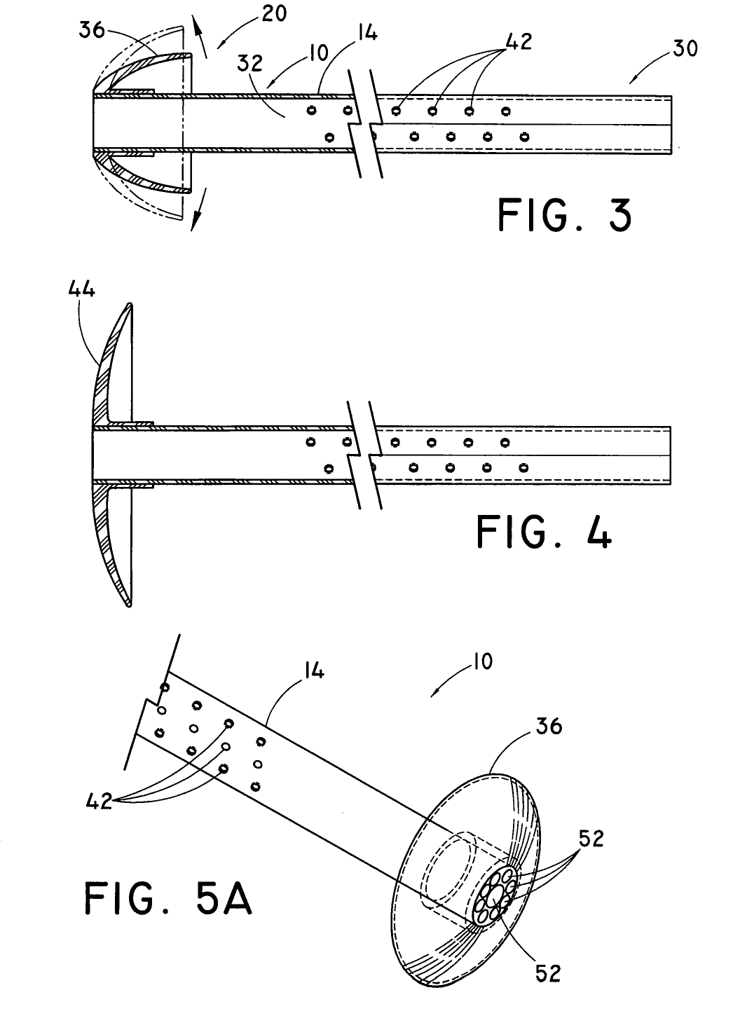

FIGS. 1A, 1B and 2 illustrate a stent 10 in accordance with embodiments of the present invention. The stent 10 includes a non-expandable, generally tubular body 14 having a proximal portion 20 and a distal portion 30. The tubular body 14 extends longitudinally along axis A as shown in FIG. 1A. A lumen 32 extends through at least a portion of the tubular body 14 of the stent 10 between a proximal opening 21 and a distal opening 31. The proximal portion 20 includes a cap portion 36 and is configured for placement proximal to the pancreatic duct as described in more detail below. One or more retaining members 38 may be included on the tubular body 14. The retaining members, when present, are configured to help hold the stent 10 in position within the pancreatic duct. One or more openings 42 may be included in the tubular body 14 as shown in FIG. 3. The openings 42 are configured to facilitate drainage through the stent 10.

As shown in FIGS. 1A and 1B, the cap portion 36 extends generally radially outward from the tubular body 14. The cap portion 36 may be curvilinear and sized and shaped to cover the opening of the pancreatic duct when the stent 10 is placed within the pancreatic duct. The cap portion 36 may be any shape such as the circular shape show in FIGS. 1A and 1B or any shape that does not irritate the biliary tree. The cap portion 36 is also sized and shaped so that the stent 10 cannot entirely enter the pancreatic duct and become trapped therein. The cap portion 36 is generally intended to remain external to the pancreatic duct in a bodily cavity and protect the opening to the duct when the tubular body 14 of the stent 10 is placed within the pancreatic duct. The cap portion 36 includes a proximal face 37 shown in FIG. 1A and a distal face 39 shown in FIG. 1B. The distal face 39 is configured to abut the tissue surrounding the opening of the duct. The proximal face 37 is configured to deflect an elongate medical device away from the pancreatic duct and also to inhibit accidental entry of contrast dye into the duct. The cap portion 36 may be domed, having a raised central portion 44 in relation to the periphery 46. The domed central portion 44 may be centrally positioned on the cap portion 36 or may be asymmetric. The domed central portion 44 may be tapered in a conical or rounded shape to help deflect the wireguide for entering the bile duct as described below. In some embodiments, the cap portion 36 may be generally flat in the central portion 44 as shown in FIG. 4. The flat central portion 44 will also be configured to help deflect the wireguide for entering the bile duct and not the pancreatic duct. The periphery 46 of the cap portion 36 extends outward to the extent that the cap portion 36 covers the opening of the pancreatic duct. The periphery 46 may be larger than the opening at the pancreatic duct, but should not be so large that the cap portion 36 interferes with entry into the common bile duct. In some embodiments, the cap portion 36 may include a one-way valve in the proximal opening 21. The one-way valve may be any kind of valve known to one skilled in the art that will allow pancreatic fluid to drain from the pancreatic duct and will inhibit contrast dye from entering the pancreatic duct once the stent 10 is in position. By way of non-limiting example, the cap portion 36 may have a diameter of about 3-5 mm. Other size diameters for the cap portion 36 are also possible.

The proximal portion 20 may include an angled portion 22 that extends at an angle away from the longitudinal axis A as shown in FIG. 1B so that the proximal opening 21 is offset from the axis A. This configuration with the angled proximal portion 22 will also help to inhibit entry of the elongate medial device or contrast dye into the pancreatic duct when the prophylactic stent 10 is in position in the duct. As discussed in more detail below, the stent 10 may be deformable for delivery so that the proximal angled portion 22 may be aligned with the distal portion 30 so that the stent 10 may be delivered over a wire guide. The stent 10 is deformable so that the stent 10 will resume the configuration shown in FIG. 1B with the angled portion 22 angled away from the axis A and the opening 21 offset from the axis A when the stent 10 is positioned in the duct in the pancreas.

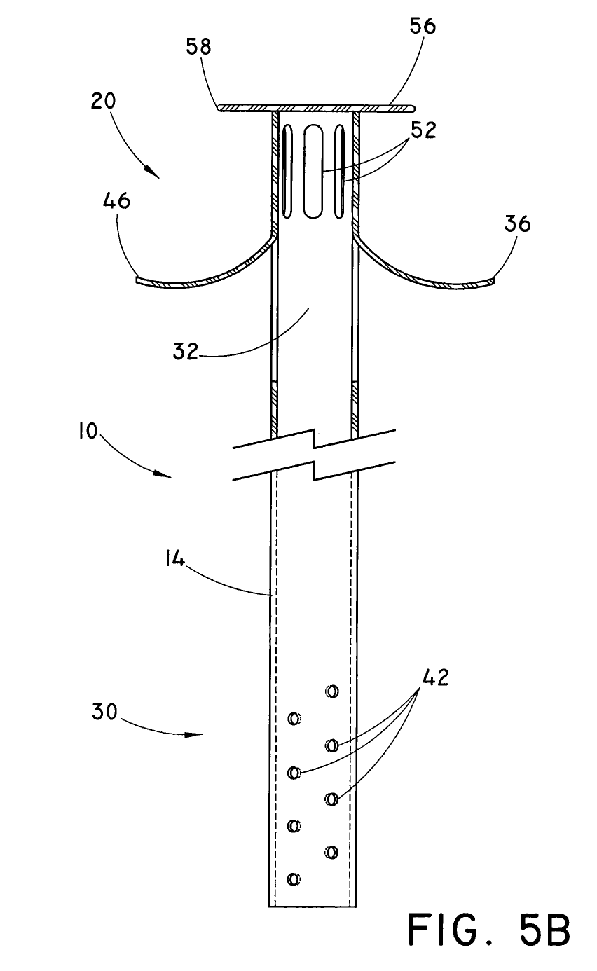

The tubular body 14 and the cap portion 36 are configured to facilitate prophylactic protection and drainage of the pancreatic duct into the duodenum. To help facilitate drainage of the pancreatic duct, tubular body 14 and the cap portion 36 may include one or more openings, i.e. the openings 42 in the tubular body as described above. The lumen 32 may extend though the body 14 to the cap portion 36, for example in by extending to an opening 52 in the central portion 44 of the stent 10. The cap portion 36 may include a plurality of openings 52. For example as shown in FIG. 5A, the cap portion 36 may include the central opening 52 surrounded by a plurality of openings 52 operably connected to the lumen 32 to facilitate drainage from the pancreatic duct. As shown in FIG. 5B, the plurality of openings 52 may be included in the tubular body 14 above the cap portion 36 and further include a cover 56 that covers the central lumen 32 to protect against accidental entry of a wire guide into the stent 10 any yet still allow drainage of the pancreatic duct through the stent 10 and into the duodenum. In the embodiment shown in FIG. 5B, the cap portion 36 is sized and shaped to cover and fit against the opening of the pancreatic duct and the cover 56 is sized and shaped to protect the lumen and the entry to the pancreatic duct yet allow drainage from the stent 10 and also allow access to the common bile duct. The cover 56 may have a shorter peripheral extension 58 than the periphery 46 of the cap portion 36. The cap portion 36 and the cover 56 are flexible and expandable so that the cap portion 36 may be collapsed into a folded configuration for delivery and an expanded configuration when the stent 10 is positioned at the pancreatic duct. The cap portion 36 is also flexible so that the cap portion 36 flexes with the movement of the body and the risk of irritation at the implant site is minimized.

In some embodiments, as described above, one or more retaining member 38 may extend outward from the tubular body 14 to help keep the stent 10 in position within the pancreatic duct. The retaining member 38 may be a flap that extends a length of about 4-8 mm from the tubular body 14. Other lengths for the retaining member may be possible and may depend on the size of the duct opening, the flexibility of the retaining member, the length of the stent and the amount of time the stent 10 is to remain implanted within the duct. The retaining member 38 may be formed from the tubular member 14 with a longitudinal cut in the wall of the tubular member 14. Alternatively, the retaining member may be formed by molding with the body 14 or addition to the tubular body 14 or any method known to one skilled in the art. The retaining member 38 may be configured for retention of the distal portion 30 of the stent 10 within the duct for several days or weeks and then allow the stent 10 to pass naturally out of the duct.

When two or more retaining members 38 are provided, the retaining members 38 may be provided circumferentially or otherwise spaced around the stent 10. For example, a first retaining member 38 may extend radially outward at a direction about 180° opposite the direction of the curved portion 36 as shown in FIG. 1. A second retaining member 38 may extend radially outwardly at about 180° from the first retaining member 38. The retaining member 38 may be sufficiently flexible to collapse against the tubular body 14 for delivery of the stent 10 and yet have sufficient resiliency to contact the pancreatic duct to hold the stent 10 in place once positioned at the delivery site.

The stent 10 may be of any size suitable for implantation into the pancreatic duct and will vary depending on the size of the duct. The stent 10 may have an outer diameter of about 3-10 Fr. The inner diameter of the stent 10 may be about 1-2 mm. Shorter or longer stents may also be used. These sizes are merely exemplary and other sized may be used. The stent may be made from materials so that the stent is soft enough to conform to the curvature of the duct and eliminate or reduce irritation at the implantation site that occurs with a rigid stent, thus reducing the risk of pancreatitis, morphological or ductal changes. The materials should also have sufficient strength to maintain a lumen through the stent when the stent is positioned within the duct. In some embodiments, the stent 10 or a portion thereof may be made from biodegradable materials. Exemplary materials for the stent 10 include, but are not limited to the following, SOF-FLE^{X}™, a type of polyether urethane, silicone, block co-polymers, urethanes, polyethylene, polystyrene, polytetrafluoroethylene (PTFE), FEP and the like and combinations thereof. In some embodiments, the stent 10 may be formed from biodegradable materials. A number of bioabsorbable homopolymers, copolymers, or blends of bioabsorbable polymers are known in the medical arts. These include, but are not necessarily limited to, polyesters including poly-alpha hydroxy and poly-beta hydroxy polyesters, polycaprolactone, polyglycolic acid, polyether-esters, poly(p-dioxanone), polyoxaesters; polyphosphazenes; polyanhydrides; polycarbonates including polytrimethylene carbonate and poly(iminocarbonate); polyesteramides; polyurethanes; polyisocyantes; polyphosphazines; polyethers including polyglycols polyorthoesters; expoxy polymers including polyethylene oxide; polysaccharides including cellulose, chitin, dextran, starch, hydroxyethyl starch, polygluconate, hyaluronic acid; polyamides including polyamino acids, polyester-amides, polyglutamic acid, poly-lysine, gelatin, fibrin, fibrinogen, casein, collagen. Exemplary non-expandable, tubular stents that may be modified for use with a cap portion include, but are not limited to, a ST-2 SOEHENDRA TANNENBAUM® stent, a COTTON-LEUNG® stent, a COTTON-HUIBREGTSE® stent, a GEENEN® Pancreatic Stent, a JOHLIN® Pancreatic Wedge Stent, or a ZIMMON® Pancreatic (available from Cook Endoscopy, Inc., Winston-Salem, N.C.).

The cap portion 36 may be formed from the same or different materials from the body 14. For example, the cap portion 36 may be formed from polyurethane while the tubular body 14 is formed from polyethylene. Alternatively, the stent 10 may be formed from unitary construction, i.e. where the entire stent if formed from silicone or polyurethane. In some embodiments, the cap portion 36 may include metallic wires within the flexible material. The wires may be formed from any metal known to one skilled in the art that is compatible with implantation and are flexible enough to allow the cap portion 36 to move between the expanded and un-expanded configurations. The cap portion 36 may be connected to the tubular body 14 using any method known to one skilled in the art, for example bonding welding and the like. The cap portion 36 may also be integrally formed with the tubular body 14 when the portions are formed from the same material. The cap 36 may also include a coating to facilitate deflection of the medical device away from the proximal opening 21 of the stent 10. For example, the cap portion 36 may include a coating to increase lubricity. Alternatively, the cap portion 36 may be formed from a material having an increased lubricity. By way of non-limiting example, the lubricious material may be selected from the following: polymers produced from monomers selected from ethylene oxide; 2-vinyl pyridine; N-vinylpyrrolidone; polyethylene glycol acrylates such as mono-alkoxy polyethylene glycol mono(meth)acrylates, including mono-methoxy triethylene glycol mono (meth)acrylate, mono-methoxy tetraethylene glycol mono (meth)acrylate, polyethylene glycol mono (meth) acrylate; other hydrophilic acrylates such as 2-hydroxyethylmethacrylate, glycerylmethacrylate; acrylic acid and its salts; acrylamide and acrylonitrile; acrylamidomethylpropane sulfonic acid and its salts, cellulose, cellulose derivatives such as methyl cellulose ethyl cellulose, carboxymethyl cellulose, cyanoethyl cellulose, cellulose acetate, polysaccharides such as amylose, pectin, amylopectin, alginic acid, and cross-linked heparin

In some embodiments, the stent 10 may include radiopaque markings (not shown) to help with placement of the stent 10 in the duct. In some embodiments, the stent 10 itself may be radiopaque. Some embodiments may include visual markings created by a laser or ink that may be visualized so the stent 10 may be visualized using fluoroscopy or x-ray. Any type of visualization marking known to one skilled in the art may be used with the stent 10.

The stent 10 may be delivered to the pancreatic duct when the physician accidently enters the pancreatic duct with a wireguide when the intended target is actually the bile duct. As discussed above, navigation with a wireguide and a catheter within the biliary tree is difficult. To avoid additional accidental entries into the pancreatic duct during the procedure and to reduce the risk of pancreatitis due to the accidental entry or repeated entry into the pancreatic duct, the wireguide may be left in position within the pancreatic duct and a stent 10 of the present invention may be delivered over the wireguide to the pancreatic duct. The stent 10 may be positioned within the pancreatic duct so that the cap portion 36 prevents further irritation of the pancreatic duct in the event of additional entries into the duct with the wire guide or accidental injection of contrast dye into pancreatic duct. The cap portion 36 is positioned to protect the pancreatic duct and to deflect the wireguide toward the bile duct. In the event the wireguide accidentally enters the lumen 32 of the stent 10, the tubular body 14 is protective of the duct and the wireguide can be withdrawn and redirected without irritating the pancreatic duct.

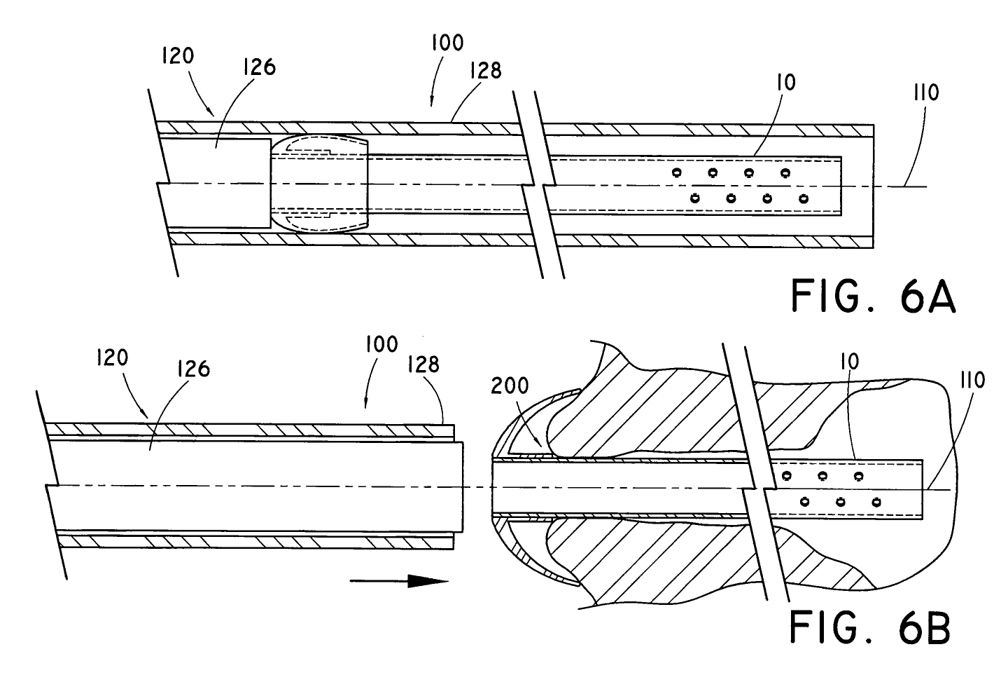

The stent 10 may be delivered to the implantation site over the wireguide that accidentally entered the pancreatic duct using any delivery system known in the art. The delivery system used will depend on the size of the stent 10 and the materials used to form the stent 10. The delivery system 100 includes a wireguide 110 that has already entered the pancreatic duct and an introducer catheter 120 that can be advanced over the wireguide 110. The stent 10 is positioned within the introducer catheter 120 as shown in FIG. 6A. The introducer catheter 120 may include a pushing catheter 126 and an outer sheath 128 for delivering the stent 10 with the cap portion in the unexpanded configuration for navigation of the catheter to the site. During delivery to the site, the stent 10 is placed over the wireguide 110 and the cap portion 36 of the stent 10 is in the unexpanded configuration. Once the introducer catheter 120 has been removed from the stent 10 at the delivery site, the cap portion 36 assumes the expanded configuration shown in FIG. 6B to cover the opening of the pancreatic duct 200. The wireguide 110 and the introducer catheter 120 have lengths sufficient to extend from the desired location in the patient's body to the exterior of the patient, as will be understood by one skilled in the art. The delivery system 100 may also include additional lumens.

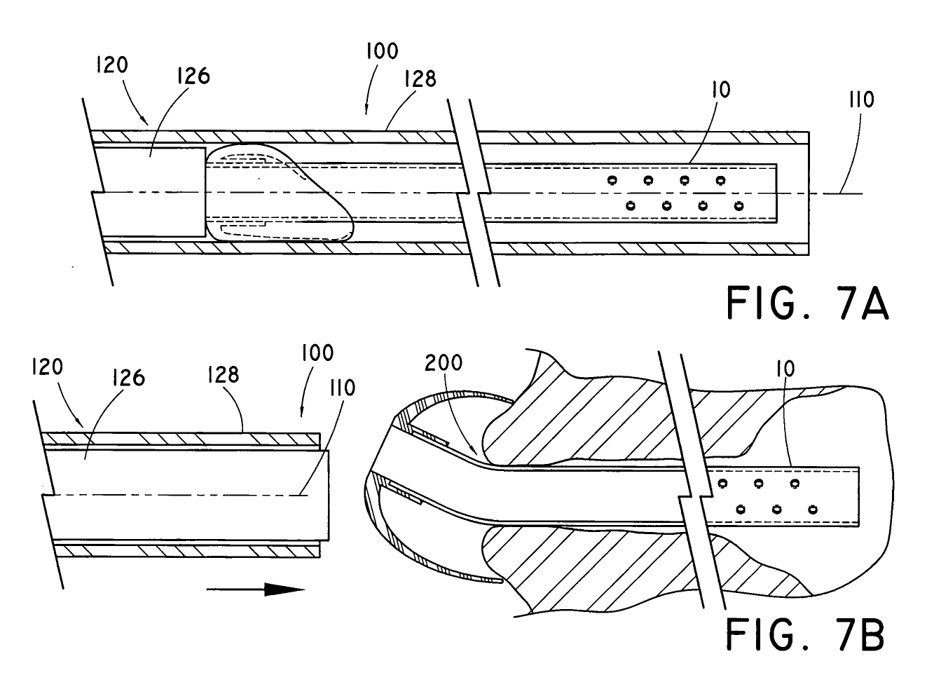

The stent 10 shown in FIG. 1B having an angled proximal portion 22 and a proximal opening 21 offset from the axis A may also be delivered using delivery systems known in the art such as the delivery system 100 shown in FIGS. 6A and 6B. The positioning of the stent 10 is shown in FIGS. 7A and 7B, where the lumen 32 extends along the wire guide 110 within the introducer catheter 120. FIG. 7B illustrates the angled proximal portion 22 returned to the angled configuration after the wireguide 110 and the introducer catheter 120 are removed from the stent 10.

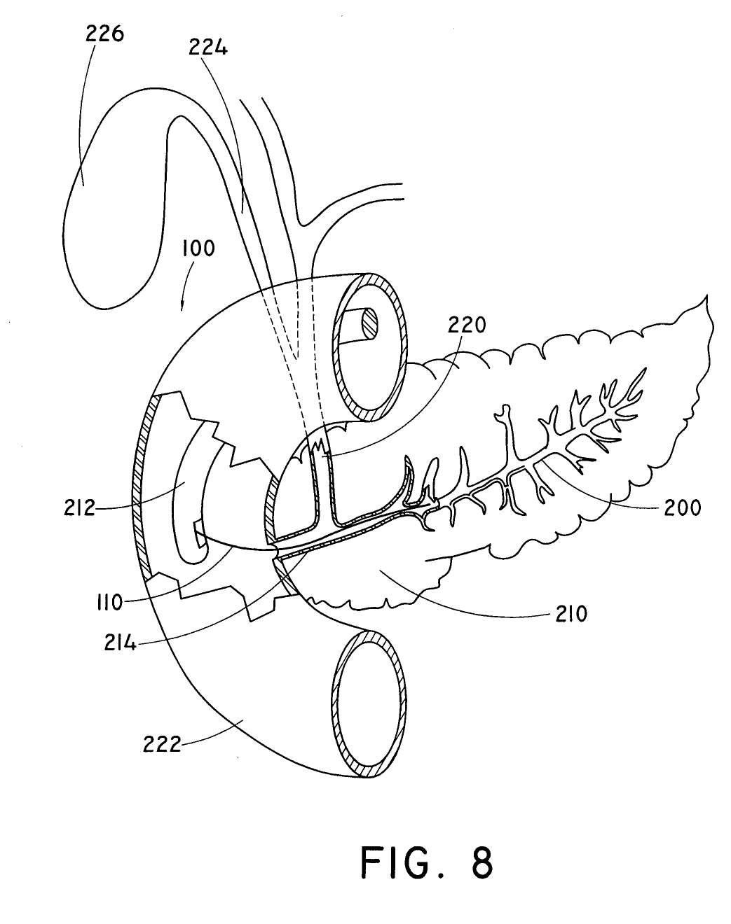

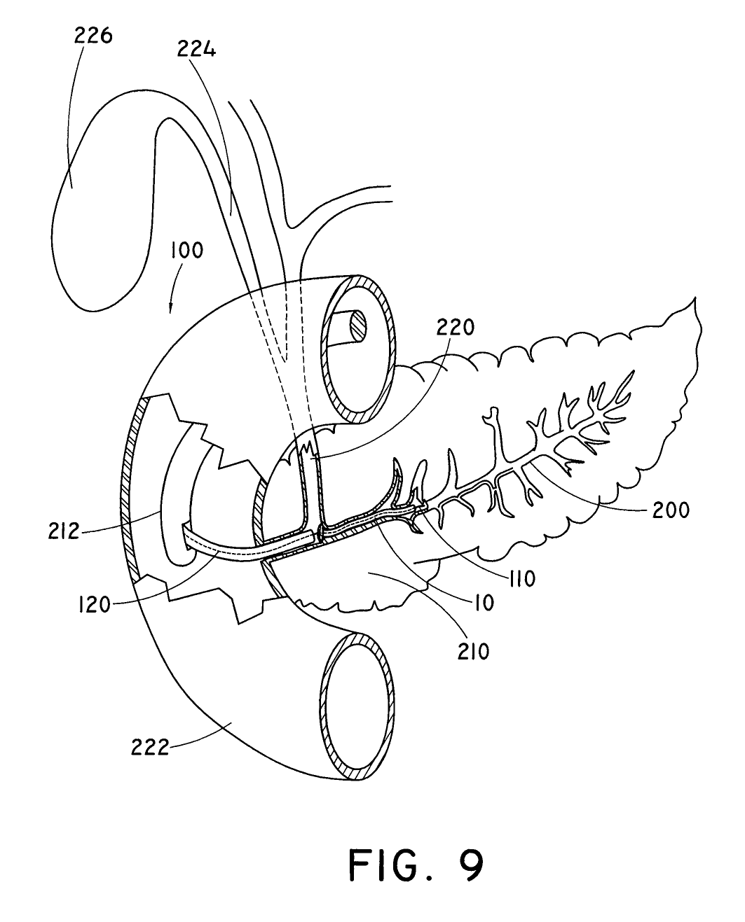

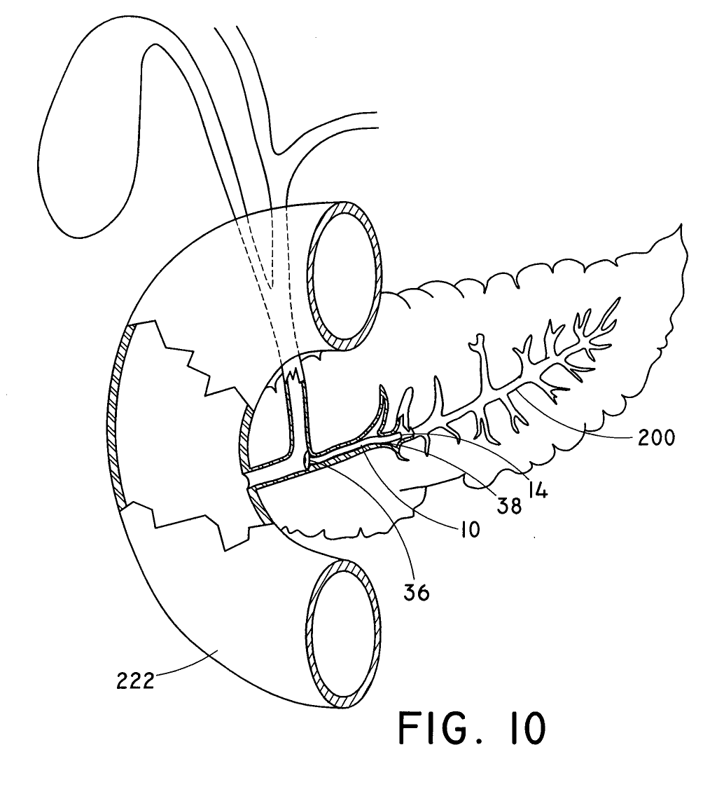

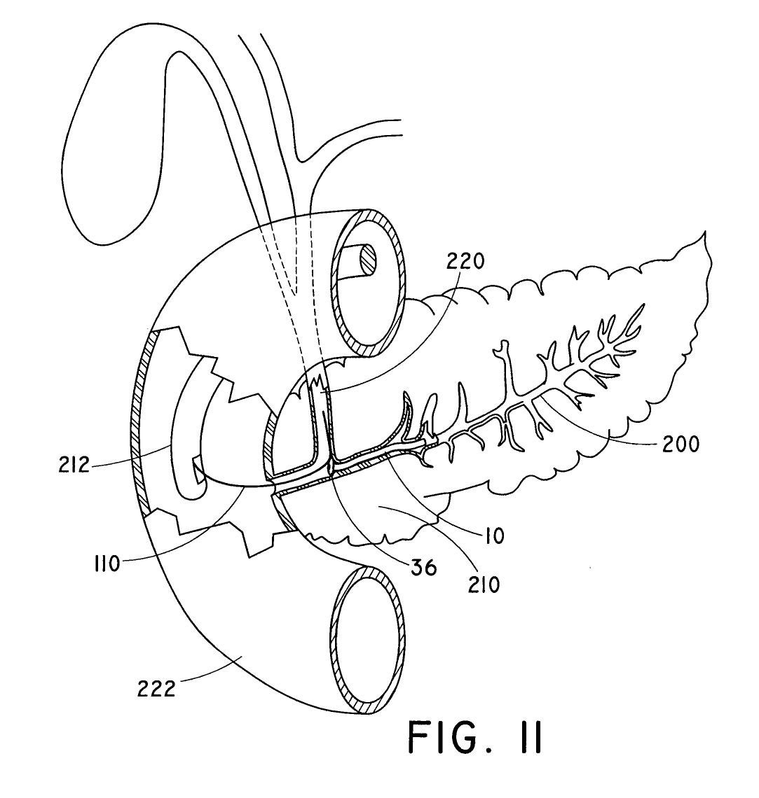

An exemplary method of delivering and implanting the stent 10 of the present invention will be illustrated with reference to the delivery system 100. As shown in FIGS. 8-11, the delivery system 100 may be used to place the stent 10 in the pancreatic duct 200 of the pancreas 210. As shown in FIG. 8, the wireguide 110 has been accidently advanced from an endoscope 212 through the Ampulla of Vater 214 into the pancreatic duct 200 instead of the common bile duct 220. Before withdrawing the wireguide 110 from the pancreatic duct 200 and attempting to enter the bile duct, the stent 10 may be positioned within the pancreatic duct 200 to prophylactically protect the duct 200 from further irritation and damage. The wireguide 110 is left in the duct 200 and the stent 10 is advanced over the wireguide 110 and out of the endoscope 212 by the introducer catheter 120. For reference, the duodenum 222, the cystic duct 224 and the gall bladder 226 are also shown. The introducer catheter 120 advances the stent 10 into position over the wireguide 110 by pushing the stent 10 distally along the wireguide 110 until the pancreatic duct 200 is reached. The stent 10 may be advanced until the tubular body 14 is positioned within the duct 200. The cap portion 36 is released from the catheter 120 and assumes the expanded configuration to cover the opening of the duct 200 as shown in FIG. 9. The cap portion 36 is positioned external to the duct 200 and covers the opening of the duct 200. The wireguide 110 is withdrawn from the duct 200 and the stent 10 is left in position as shown in FIG. 10 to prophylactically protect the pancreatic duct 200 from further irritation and entry by the wireguide 110 or from accidental injection of contrast dye. FIG. 11 illustrates the wireguide 110 advancing through the Ampulla of Vater 214 and deflecting off the cap portion 36 of the stent 10 and entering into the bile duct 220.

The stent 10 may remain in position for up to about two weeks. Shorter or longer periods of implantation for the stent 10 are also possible. The stent 10 may be pushed out of the duct 200 by natural body function or the stent 10 may be surgically removed. Where the stent 10 or portions thereof are biodegradable, the stent 10 will degrade and be passed by the body. In some embodiments, the tubular body 14 may be degradable and the cap portion 36 may not be degradable. In that instance, the tubular body 14 may degrade so that the cap portion 36 falls off into the duodenum 222 and is passed by the body.

The above Figures and disclosure are intended to be illustrative and not exhaustive. This description will suggest many variations and alternatives to one of ordinary skill in the art. All such variations and alternatives are intended to be encompassed within the scope of the attached claims. Those familiar with the art may recognize other equivalents to the specific embodiments described herein which equivalents are also intended to be encompassed by the attached claims. For example, the invention has been described in the context of the biliary system for illustrative purposes only. Application of the principles of the invention to any other bifurcated lumens or vessels within the body of a patient, including areas within the digestive tract such as the pancreatic system, as well as areas outside the digestive tract such as other vascular systems, by way of non-limiting examples, are within the ordinary skill in the art and are intended to be encompassed within the scope of the attached claims.

1. A prophylactic stent for inhibiting unintentional entry of an elongate medical device into a passageway of an internal bodily duct of a patient, the stent comprising:
a non-expandable, generally tubular body having a proximal portion and a distal portion, a lumen extending through at least a portion of the body; a distal opening in the distal portion in fluid communication with the lumen, and a proximal opening in the proximal portion in fluid communication with the lumen, the body being configured to be disposed within the internal bodily duct and facilitate the passage of bodily fluid therethrough;
a cap portion operably connected to the proximal portion of the body, the cap portion movable between a non-expanded configuration and an expanded configuration, the non-expanded configuration having an expanded diameter configured to protect an opening to the internal bodily duct so as to prevent an unintentional entry of an elongate medical device through the opening and into the passageway of the internal bodily duct.

2. The stent of claim 1, wherein the cap portion is configured to secure the proximal portion of the body in an internal bodily cavity adjacent to the internal bodily duct, the internal bodily cavity being in fluid communication with the internal bodily duct via the opening, the internal bodily cavity having a cross-sectional area that is greater than that of the internal bodily duct.

3. The stent of claim 1, wherein the cap portion includes a proximal face and a distal face, the distal face configured to engage tissue surrounding an entry to the passageway and the proximal face configured to deflect the elongate medical device.

4. The stent of claim 1, wherein the proximal opening extends along an axis angled away from a longitudinal axis of the distal portion to inhibit unintentional entry of the elongate medical device through the proximal opening.

5. The stent of claim 1, further comprising a retaining member extending outward from the tubular body.

6. The stent of claim 1, wherein the cap portion further comprises a plurality of openings operably connected to the lumen.

7. The stent of claim 1, wherein the cap portion comprises a domed portion.

8. The stent of claim 1, wherein the tubular body comprises a plurality of body openings operably connected to the lumen.

9. The stent of claim 1, wherein an outer diameter of the tubular body is between about 3 French to about 10 French.

10. The stent of claim 1 wherein the cap portion has a diameter of about 3-5 mm.

11. The stent of claim 1, wherein the stent comprises a material selected from the group consisting of plastics, silicone, block polymers, urethanes, polystyrene, polyethylene, PTFE, FEP and combinations thereof.

12. The stent of claim 1, wherein the cap portion comprises a lubricous material.

13. The stent of claim 1, further comprising a cover portion at a proximal end of the stent, the cover portion configured to protectively cover openings formed in the tubular body above the cap portion, wherein the cover allows drainage through the openings.

14. The stent of claim 1, further comprising a one-way valve in the proximal opening, the one-way valve configure to allow pancreatic fluid out from the pancreatic duct and to inhibit fluid flow into the pancreatic duct.

15. A method for implanting a prophylactic stent in a duct, the method comprising:
entering a branched ductal system with a wireguide;
advancing the wireguide into a first duct of the branched ductal system;
advancing a stent over the wireguide, the stent comprising:
a non-expandable, generally tubular body having a proximal portion and a distal portion, a lumen extending through at least a portion of the body; a distal opening in the distal portion in fluid communication with the lumen, and a proximal opening in the proximal portion in fluid communication with the lumen;
a cap portion operably connected to the proximal portion of the body, the cap portion movable between a non-expanded configuration and an expanded configuration, the non-expanded configuration having an expanded diameter;
implanting at least a portion of the distal portion of the stent in the first duct so that the cap portion is in the expanded configuration and protecting an opening in the first duct to inhibit the wireguide from reentering the first duct.

16. The method of claim 15, further comprising withdrawing the wireguide from the first duct and advancing the wireguide into a second duct of the branched ductal system, wherein the cap portion inhibits the wireguide from advancing into the first duct.

17. The method of claim 15, wherein the stent is advanced in a straight configuration wherein the proximal portion and the distal portion are substantially aligned along a longitudinal axis.

18. The method of claim 17, wherein the stent is implanted in an angled configuration wherein the proximal portion is angled away from the longitudinal axis and the proximal opening is offset from the longitudinal axis.

19. The method of claim 15, wherein the cap portion is implanted in the expanded configuration in a bodily cavity having a cross-sectional area that is greater than the opening of the duct.

20. The method of claim 15, comprising implanting the portion of the distal portion of the stent in the pancreatic duct.
